(19) United States
(12) Patent Application Publication
(10) Pub. No.: US 2016/0287487 A1
(43) Pub. Date: Oct. 6, 2016

(54) Fragrance slurry pattern providing controlled release of the fragrance Aroma

(71) Applicants: Sven Dobler, Huntington, NY (US); James Mabry, Chattanooga, TN (US); Charles Whitaker, Chattanooga, TN (US); Douglas Whitaker, Chattanooga, TN (US); Ken Maury, Chattanooga, TN (US)

(72) Inventors: Sven Dobler, Huntington, NY (US); James Mabry, Chattanooga, TN (US); Charles Whitaker, Chattanooga, TN (US); Douglas Whitaker, Chattanooga, TN (US); Ken Maury, Chattanooga, TN (US)

(21) Appl. No.: 14/999,688

(22) Filed: Jun. 15, 2016

Related U.S. Application Data
(63) Continuation-in-part of application No. 12/807,416, filed on Sep. 3, 2010, now abandoned.
Application No. 12/807,416 is a continuation of application No. 11/438,237, filed on May 22, 2006, now abandoned.
(60) Provisional application No. 62/231,011, filed on Jun. 22, 2015.
(60) Provisional application No. 60/683,979, filed on May 24, 2005.

(51) Int. Cl. A61K 8/02; A61Q 13/00

(52) U.S. Cl. CPC A61K 8/0216; A61Q 13/00

(57) ABSTRACT

A fragrance slurry pad patterned sampler including a base material applied to one of coated and uncoated paper, flexible film and paper laminations, aluminum foil, thermal plastics, PET, OPP, BOPP, EVA, PE, and other commonly polymer films providing the base for the fragrance sampler, the sampler including a slurry formed from various aroma chemicals, one sampler formed of one or more aroma chemicals, and the other sampler formed of one or more aroma chemicals, each applied to the base material, in a patterned arrangement, so that when the microencapsulated chemicals are fractured upon opening of the sampler, the separate aromas will be delivered sequentially, or in combination, depending upon their patterned application to the base sheet of the sampler.

### CROSS REFERENCE TO RELATED APPLICATIONS

This non-provisional patent application claims priority to the provisional patent application having Ser. No. 62/231,011 filed on Jun. 22, 2015, and which claims priority to the continuation-in-part patent application having Ser. No. 12/807,416, filed on Sep. 3, 2010; and this application is a continuation of the application having Ser. No. 11/438,237, filed on May 22, 2006, and which application claims priority to the provisional patent application having Ser. No. 60/683,979. filed on May 25, 2005.

### BACKGROUND OF THE INVENTION

This fragrance slurry pad relates to generally perfume or fragrance sampling devices, and more specifically pertains to a pad that may have a patterned application of aroma chemicals, generally microencapsulated, so that when the sampler is pulled open, there is a selective fracturing of the patterned fragrances in order to provide the specific aroma sought by the retailer, and their customers, or for delivery through magazines, scent cards, and other related devices.

Microencapsulated fragrances have been used for decades, for the purpose of sampling fragrances, and many of these have applications upon fragrance strips, upon scent cards, and related types of delivery means. Usually, these types of products generally involve the application of a microencapsulated fragrance “slurry” on the surface of a coated or uncoated text or cover stock weight paper, then drying of the slurry by means of heated forced air, or air drying, and then allowing the consumer to subsequently liberate the fragrance by either scratching or breaking of the microcapsules, delivering the scented aroma to the ambient air. In addition, fragrance strip products generally involve the application of microencapsulated slurry in the fold area of a coated text weight paper, allowing the slurry to dry and adhere to the inside surfaces of the folded paper area by means of absorption and evaporation of the slurry carrier material. The capsules break and liberate the fragrance upon opening the fold area of the paper. Microencapsulation has been well documented in the chemistry and printing literature. The carrier systems for the encapsulated fragrances are predominately hydrous but synthetic capsules in anhydrous, solvent based systems using such materials as urea-formaldehyde, are also common in the industry.

Usually, the above-mentioned fragrance sampling products can be manufactured on lithographic, flexographic, or rotogravure printing machines, both in sheet or roll form, using silk screen, flexography, extrusion and airless sprayed technology among other methods to apply the fragrance slurry. Manufacturers of fragrance strips have predominately used flexographic printing technology to apply the slurry during heat-set, off-set web printing. Flexography involves raised printing, where the slurry is transferred from the raised surface contour of a printing plate or pad to a paper. Plates and pads are generally made using photo technology, by etching nontransfer areas away from a soft material such as rubber using mechanical, laser and other technology, dye molding, casting, extruding and forming liquid, heated or resinous materials.

Fragrance strips have become the predominant product used in fragrance sampling in this Country, with estimates ranging between 2.5-3.5 billion units annually. Fragrance strips appear in a variety of common print format including periodical and national magazine inserts, direct mail, catalogue blow-in and stitch-in inserts, billing statement enclosures, mail order envelopes and business response envelopes and billing statements and remittent envelopes. Consumers and readers of magazines generally appreciate and enjoy receiving free fragrance samples, but, the competing fragrance marketing messages have become almost overwhelming. Often more than three fragrance strips are included in mail such as subscription magazines, catalogues, direct mail, and store and credit card billing statements.

The essence of the current invention is to provide a more controlled application of such fragrance sampling onto the identified sampling devices, but to provide for a patterned application of a variety of fragrances so that either the delivery of the aroma can be done sequentially, of different fragrances, or where a combination of the fragrances may be microencapsulated onto the sample, and fractured and deliver aromas in combination, to arrive at that specific aroma desired by the manufacturer, and deliver to the consumer, in order to encourage their purchase of the identified fragrance, from the market.

There are a variety of prior patents that disclose the early delivery of liquids, and fragrances, from samplers.

For example, the patent to Davis, U.S. Pat. No. 3,427,277, describes a method for forming a pressure sensitive liquid dispensing device, and contains a reservoir, and a liquid, mixed with other ingredients, providing a stable gel-like structure used for coloring or inking materials.

The patent to Dreger, U.S. Pat. No. 4,769,264, shows an on-page fragrance sampling device. It also discloses the use of an adhesive layer containing microcapsules of fragrance.

The patent to Gunderman, et al, U.S. Pat. No. 5,566,693, also discloses a fragrance sampler. This patent describes a silk screen method for applying a perimeter adhesive, not by a flexographic method, which also includes a protective thin film overlay to protect the applied fragrance.

The patent to Clancy, et al, U.S. Pat. No. 4,100,853, discloses a method of forming a porous shaped body capable of retaining liquids therein. Another patent to Gunderman, et al, U.S. Pat. No. 5,690,130, discloses a cosmetic sampler with integral applicator.

Other patents disclosing air freshening devices, and the like, includes the patents to Dutcher, U.S. Pat. No. 4,208,012, and patent U.S. Pat. No. 4,155,500. Most of these devices relate to cartons that hold air freshener compositions.

A greeting card that produces an odor when opened is shown in the U.S. patent to Norfoleet, U.S. Pat. No. 4,484,768. A fragrance-releasing pull-apart is shown in the patent to Turnbull, et al, U.S. Pat. No. 4,487,801. A fragrance-releasing microcapsules on a see-through substrate is shown in the patent to Sweeny, et al, U.S. Pat. No. 4,493,869. An on-page fragrance sampling device is shown in the patent to Charbonneau, et al, U.S. Pat. No. 4,606,956.

Another pad fragrance sampling device is shown in the patent to Charbonneau, patent U.S. Pat. No. 4,661,388. A patent to Moir, et al, U.S. Pat. No. 4,751,934, discloses a cosmetic sampler. A patent upon a perfume patch is shown in the patent to Scycher, et al, U.S. Pat. No. 4,880,690. A sampler and method of making same is shown in U.S. Pat. No. 5,161,688, to Muchin. Another patent to Moir, et al, U.S. Pat. No. 5,192,386, discloses a method of making a cosmetic sampler. This type of a sampler discloses the formation of a well, within a paper stock, and into which the cosmetic sample, powder of solvent may locate.

Pouch type samplers can be seen in the patent to Bootman, et al, U.S. Pat. No. 5,391,420, in addition to the patent to Kasianovitz, et al, U.S. Pat. No. 5,616,337.

The patent to Dobler, one of the inventors herein, shows a fragrance sampler insert, in U.S. Pat. No. 6,461,620.

Another fragrance packet sampler is shown in the patent to Witenger and Dobler, two of the inventors herein, in their U.S. Pat. No. 5,645,161.

These are examples of what the prior art discloses relating to fragrance samplers, and upon which the current invention improves.

### SUMMARY OF THE INVENTION

This invention relates to the application of a designed pattern of specific and different aroma of fragrances onto a carrier such that when the various microencapsulated fragrances are fractured, the aromas will either sequentially or in combination produce a desired array of aromas to the delight of the user and develop a desire for usage of a selective fragrance, as generated.

There are previous applications of fragrance slurry pad patterns in the prior art, as previously described. These pads are of rubber or polymer materials, with specific profile designs that are used in flexographic glue units to apply materials, typically adhesives, coatings, or microencapsulated fragrance slurry, as to paper, typically during the web, heat-set, and off-set printed process. Frequently, these prior patterns were for decorative purposes, initially where the fragrance materials could be colored and then be printed with the pads showing raised transfer areas in the shape of a logo, mark or other discernible image. Prior to that the pads were just random patterns designed only for the functional requirements of depositing materials in certain patterns to adjust the lay down and performance of the deposited materials. As an example, if you lay down an adhesive in a pattern with 50% transfer area, the adhesion is different than if you lay down the same adhesive with a 60% transfer area. The more transfer, the more adhesion, and the area of deposit will effect how and where the adhesion occurs.

When used for fragrance sampling in the manufacture of scented strips and for magazines or catalogues, manufacturers are already using various pad designs to change the olfactive rendition of the fragrance materials deposited to the paper. The more you lay down and the pattern in which you lay down one and the same slurry will result in more or less microcapsules rupturing when opened, and the release profile, or smell, of the fragrance is impacted.

The concept of this invention recognizes that a fragrance is a complex combination of many aroma generating chemicals. Perfumers speak of top notes, heart or middle notes, and base notes. Each of these notes themselves can include dozens of aroma chemicals in combination. When picking one of the many current pad designs, manufacturers and their customers go through an evaluation process to find out which pad and which base slurry combination results in the best rendition, best in the sense of best matching the eau de toilette retail product smell. One pad design may help push the heart or the base notes, another may be the top note. But so far, all manufactures had to pick one pad in the end, for instance, just a pad that contains a diamond pattern, or a dot pattern, and compromises are therefore made therefrom.

The invention herein solves this problem. This concept can make a custom pad that recombines elements of the individual pad designs. For example, a diamond pattern combined with a dot pattern on the same pad to deliver different aspects or parts of the individual fragrances, while timing of their release, since these scented strips, as pattern imprinted, or in a fold, and the leading edge gets ruptured before the center or gutter side of the fold gets opened. Hence, the concept of this invention is to place two or more separate pads, with different patterns, either next to one another, or by combining two or more patterns together, on the same pad, to provide delivery of multi aroma fragrances, to the enticement of the sampler, and potential consumer of the delivered and controlled fragrance

The fragrance slurry pad patterned sampler includes a carrier or base material, which may be formed of coated and uncoated paper, flexible film and paper laminations, aluminum foil, thermal plastics, PET, OPP, BOPP, EVA, PE, and other common polymer films providing the base for the fragrance sampler, and upon which the slurry formed of the various aroma chemicals may be applied.

It is, therefore, the principal object of this invention to provide a combination pattern of different aroma chemicals so that unique combinations of aromas produce a fragrance that will be desired by the sampler.

Another object of this invention is to place two or more separate pads, of select aroma chemicals, into different patterns, adjacent one another upon the sampler, so as to furnish a combination of aromas, in sequence, for delivery to the sampler.

Another object of this invention is to provide a combination of two or more different patterns of aroma chemicals, where the patterns may be intermixed with each other upon the pad, so as to produce a simultaneous delivery of different aroma chemicals, for the sampler to smell, while sampling of the prepared sampler strip, card, or the like.

These are other objects may become more apparent to those skilled in the art upon review of the summary of the invention as provided herein, and upon undertaking a study of the description of its preferred embodiment, in view of the drawings.

### BRIEF DESCRIPTION OF THE DRAWINGS

In referring to the drawings:

FIG. 1 shows a scent strip having a diamond pattern of aroma chemicals applied to its scented strip;

FIG. 2 provides a view of another scent strip having a dot pattern of selective aroma chemical(s) applied thereto in preparation for its assembly into a useable scent strip or card;

FIG. 3 shows a combination of patterned applied aroma chemicals onto a pad for use in the preparation of the scent strip or card, which when applied and opened, initially fractures the microencapsulated chemicals of the dot pattern, and subsequently the diamond pattern, to produce a combination of aromas for the person applying the sampler; and

FIG. 4 shows the preparation of a scent strip containing various aroma chemicals in a combination of dot patterns and diamond patterns and which produce simultaneously the generation of a fragrance aroma, from a variety of the aroma chemicals, applied as microencapsulated fragrances onto the scent strip during its production.

### DESCRIPTION OF THE PREFERRED EMBODIMENT

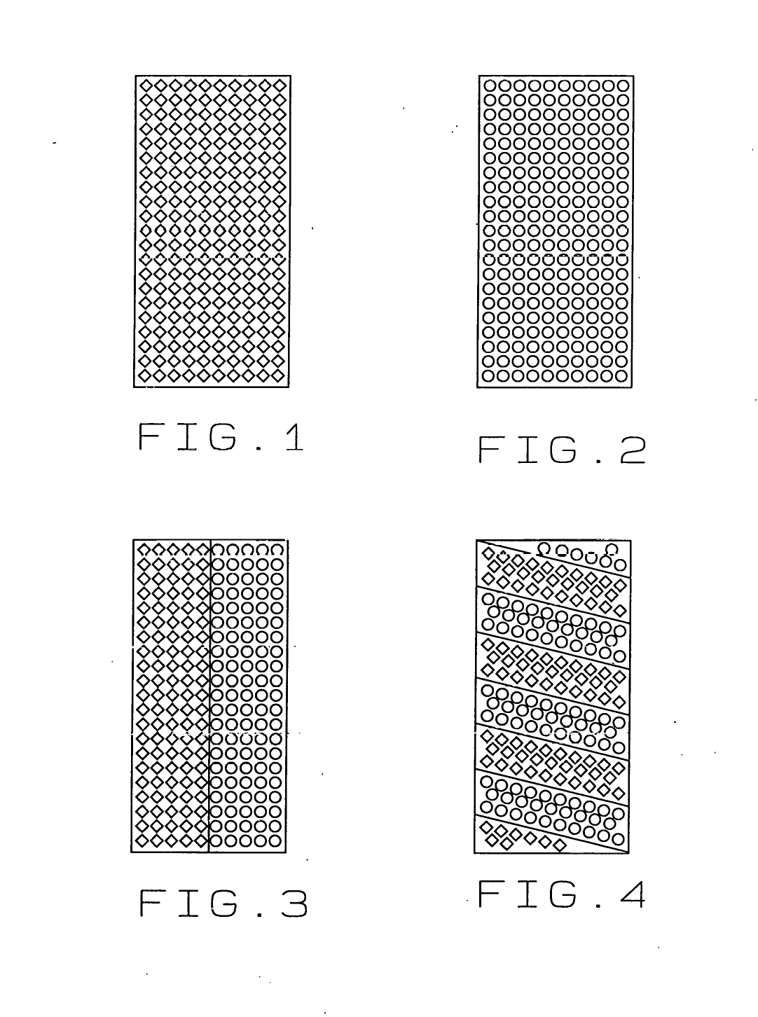

The present art enhances the usage and application of a fragrance slurry transferred by flexographic pad to paper or other substrate for use as a fragrance sampler. As can be seen in FIGS. 1 and 2, the fragrance slurry is applied in a particular geometric pattern, as can be seen in FIG. 1, as a diamond pattern, and in FIG. 2, as a dot pattern matrix upon the surface of the fragrance strip during its processing.

As is known in the art, the pickup cylinder in flexographic printing usually absorbs the fragrance slurry from its delivery pan, and that cylinder transfers it to a cylinder pad, and the raised pad on the transfer cylinder carries the slurry and transfers it to a passing web of paper, or other substrate used for forming on the sampler. Obviously, the amount of fragrance slurry transferred to the paper is controlled by web tension, the pressure between the web and the pad, and the speed of rotation of the pickup cylinder, the pad, and the speed of passage of the passing web, all of which is coordinated for the most efficient transfer of the fragrance slurry onto the sampler, during its preparation. This is explained in greater detail in the application having Ser. No. 12/807,416.

As previously explained, the various chemicals that make up a fragrance is a complex combination of many aroma chemicals. Usually these aroma chemicals are applied in laminations, the initial one identified as the base note, then you may have a heart or middle note, and then an upper layer called the top note, and all of these chemicals form the aroma of the desired fragrance preferred by the perfumer designer.

The object of this current invention is to provide a means for controlling the release of the microencapsulated fragrances, or aromas, either in sequence, or consecutively, through the use of a patterned design of such aroma chemicals, upon the sampler strip or card.

For example, as can be seen in FIG. 3, therein is disclosed the use of a diamond pattern of one aroma chemical, or a combination of such chemicals, in an initial pattern, as noted on the left side of the sampler, and combined with a dot pattern of the same or other aroma chemicals, on the same pad, so as to arrange the different aspects of the fragrance, and to time their separate release, as the sampler is opened, such as when it is taken from the magazine sheet, or simply pulled open, by removal of its cover, in order to fracture the microencapsulated segregated aroma chemicals for disseminating their aroma to the surrounding area, for sampling. Or, the particular patterned strip as shown in FIG. 3 may include its center arranged fold line, where the two sides of the shown strip overlay one another, and/or adhere together by peelable adhesive, that may be included within the chemical makeup of the slurry, as applied to the separate halves of the strip, such that when the overlay is separated, there is a generation of a combination of aromas, producing the desired fragrance, sought by the perfumer, when the sampler is tested by the potential consumer.

FIG. 4 shows a further patterned array of a series of aroma chemicals, in this particular instance, it can be seen that a series of dot patterns of one fragrance of microencapsulated fragrance chemical(s) may be applied intermediate rows of the separate aroma chemical(s) formulated to form the diamond pattern of aroma chemicals making up that aspect of the formulation. Thus, when an overlying cover is removed, as known in the art, the microencapsulated aroma chemicals making up both the diamond pattern, and the dot pattern, will simultaneously release their aromas, to form that generic fragrance sought by the perfumer, for testing by the potential consumer, when testing the sampler strip or card either taken from a magazine, or as furnished at the cosmetic counter of the retail store.

These are examples as to how a combination of the aroma chemicals can be combined into formulating the overall fragrance sought for the sample, and to be delivered to the consumer, when the sampler is tested. As previously explained, each fragrance is generally made up of a complex combination of many of these aroma chemicals. Each of these notes that make up the fragrance can include dozens of the aroma chemicals themselves. Many of the fragrance companies may utilize as many as 15, or more, of a combination of these aroma chemicals to make up their desired fragrance. In this particular invention, a combination of these aroma chemicals may make up the diamond pattern, and a combination of additional aroma chemicals may make up the dot pattern, and these can be released either sequentially, or simultaneously, depending upon whether a note pattern as shown in FIG. 3, or a note pattern as shown in FIG. 4, may be employed. Nevertheless, the concept of this invention is to provide the delivery of selective aroma chemicals, forming the slurry for the sampler, and apply them in combination upon the sampler, in order to obtain either that sequential or simultaneous delivery of their separate slurries for enticement of the ultimate consumer.

Variations or modifications to the subject matter of this invention may occur to those skilled in the art upon review of the invention as described herein. Such variations, if within the spirit of this invention, are intended to be encompassed within the scope of any claims to patent protection issuing hereon. The definition of the invention in the preferred embodiment, and its depiction in the drawings, are all set forth for illustrative purposes only.

I claim:

1. A fragrant slurry pad patterned sampler including a substrate, a microencapsulated fragrance material applied to said substrate, fragrance material having discrete patterned applications to the substrate, said fragrance material comprised of select aroma chemicals, at least two portions of the fragrance material applied to the substrate, one of said fragrance material as applied in an initial pattern, and a second fragrance material applied in a separate pattern, such that when the sampler is opened, the various fragrance materials releasing one of sequentially or simultaneously their separate aromas for providing a fragrance for sampling by the user.

2. The sampler of claim 1, wherein the first segment of aroma chemicals are applied to one side of the base substrate, while the second segment of the aroma chemicals are applied to an adjacent side of the base substrate of the sampler, whereby when the cover sheet is removed from the sampler, initially one segment of the sampler will deliver its fragrance to the atmosphere, and subsequently, as the cover sheet is fully removed, the second segment of the aroma chemicals will he released to the atmosphere.

3. The sampler of claim 2, and including a fold line provided between the first and second segments of the aroma chemicals, and the base substrate is folded along said fold line, such that when the sampler is opened, both segments of the aroma chemicals are released into the atmosphere simultaneously to provide its fragrance.

4. The sampler of claim 1, wherein the first segment of aroma chemicals is interspaced amongst a second segment of aroma chemicals, such that when the cover sheet is removed, the microencapsulated fragrances are released simultaneously to present their fragrance to the atmosphere.
